(19) United States
(12) Patent Application Publication
(10) Pub. No.: US 2018/0049720 A1
(43) Pub. Date: Feb. 22, 2018

(54) ULTRASOUND BEAMFORMING SYSTEM AND METHOD

(71) Applicant: Carestream Health, Inc., Rochester, NY (US)

(72) Inventor: Ajay ANAND, Rochester, NY (US)

(21) Appl. No.: 15/644,955

(22) Filed: Jul. 10, 2017

Related U.S. Application Data
(60) Provisional application No. 62/375,925, filed on Aug. 17, 2016.

(51) Int. Cl. A61B 8/08; A61B 8/00

(52) U.S. Cl. CPC A61B 8/5223; G10K 11/34; A61B 8/5246; A61B 8/466

(57) ABSTRACT

A method for ultrasound imaging acquires an initial set of one or more images of a region of interest in a predetermined clinical ultrasound mode using a generated signal of a first beamforming type. Suitability of the first beamforming type signal for imaging the region of interest in the predetermined mode is evaluated according to a metric calculated from the initial set of one or more images. The generated signal is automatically changed to a second beamforming type according to the suitability evaluation.

### CROSS REFERENCE TO RELATED APPLICATIONS

This application claims the benefit of U.S. Provisional Ser. No. 62/375,925, provisionally filed on Aug. 17, 2016, entitled “ULTRASOUND BEAMFORMING SYSTEM AND METHOD”, in the name of ANAND, which is incorporated herein in its entirety.

### TECHNICAL FIELD

The disclosure relates generally to the field of medical diagnostic ultrasound systems and methods and more particularly to apparatus and methods that help to automate ultrasound mode selection for an exam based on detected image characteristics.

### BACKGROUND

Ultrasound imaging systems/methods are known. See for example U.S. Pat. No. 6,705,995 (Poland) and U.S. Pat. No. 5,370,120 (Oppelt). All of the above-identified references are incorporated herein by reference in their entirety.

Conventional ultrasound imaging apparatus can have one or more transducers, transmit and receive beamformers, and various processing and display components used for generating and presenting the acquired images. A transmit beamformer supplies electrical waveform signals to the transducer arrays on the hand-held probe, which, in turn, generates the associated ultrasonic signals. Objects in the path of the transducer signals scatter ultrasound energy back to the transducers, which then generate receive electrical signals. The receive electrical signals are delayed for selected times specific to each transducer, so that ultrasonic energy scattered from selected regions adds coherently, while ultrasonic energy from other regions has no perceptible impact. Array processing techniques used for generating and processing received signals in this way are termed “beamforming” and are well known to those in the ultrasound imaging field.

Ultrasound systems can offer the sonographer a number of imaging options for obtaining image content suitable for patient assessment and diagnostics. Among options selectable by the operator or pre-programmed by the system designer are different types of beamforming algorithms. Selection of the beamforming sequence that best meets the requirements for a particular exam can be based on a number of factors, including imaging frame rate, relative noise levels, and various imaging characteristics.

Harmonic imaging is viewed by some as offering improvements over conventional ultrasound imaging. With conventional ultrasound beam processing, the receive filters are tuned to the range of the transmitted signal. The returned signal, attenuated by the subject tissue, is nominally close to the same frequency as the generated signal. In harmonic imaging, however, the receive filters are tuned to the range of the harmonic frequency (typically using the second harmonic, at 2× the emitted frequency) that results from the non-uniform response of the tissue under examination. The harmonic signal content can be quickly processed and used to generate image content of good quality.

It can be difficult to determine which type of beamforming will be suitable/successful under particular conditions. This disclosure is directed to medical diagnostic ultrasound imaging systems, and, more particularly, to a system and method for an on-the-fly ultrasound beamforming paradigm determination based on a data driven approach.

### SUMMARY

An object of the present disclosure is to advance the art of ultrasound imaging and overall system operation.

These objects are given only by way of illustrative example, and such objects may be exemplary of one or more embodiments of the invention. Other desirable objectives and advantages inherently achieved by the may occur or become apparent to those skilled in the art. The invention is defined by the appended claims.

According to one aspect of the disclosure, there is provided a method for ultrasound imaging comprising: acquiring an initial set of one or more images of a region of interest in a predetermined clinical ultrasound mode using a generated signal of a first beamforming type; evaluating the suitability of the first beamforming type signal for imaging the region of interest in the predetermined mode according to a metric calculated/determined/extracted from the initial set of one or more images; and automatically changing the generated signal to a second beamforming type according to the suitability evaluation.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A and 1B show exemplary ultrasound systems.

FIG. 2 shows a schematic of an exemplary ultrasound system.

FIG. 3 illustrates a Sonographer using an exemplary ultrasound system.

FIG. 4 shows a displayed ultrasound image.

FIG. 5 shows a flowchart of tissue harmonic imaging.

FIG. 6 shows a flowchart of duplex imaging and spectral Doppler evaluation.

### DETAILED DESCRIPTION OF THE EMBODIMENTS

The following is a detailed description of the preferred embodiments, reference being made to the drawings in which the same reference numerals identify the same elements of structure in each of the several figures.

Medical ultrasound (also known as diagnostic sonography or ultrasonography) is a diagnostic imaging technique based on the application of ultrasound, used to display internal body structures such as tendons, muscles, joints, vessels and internal organs.

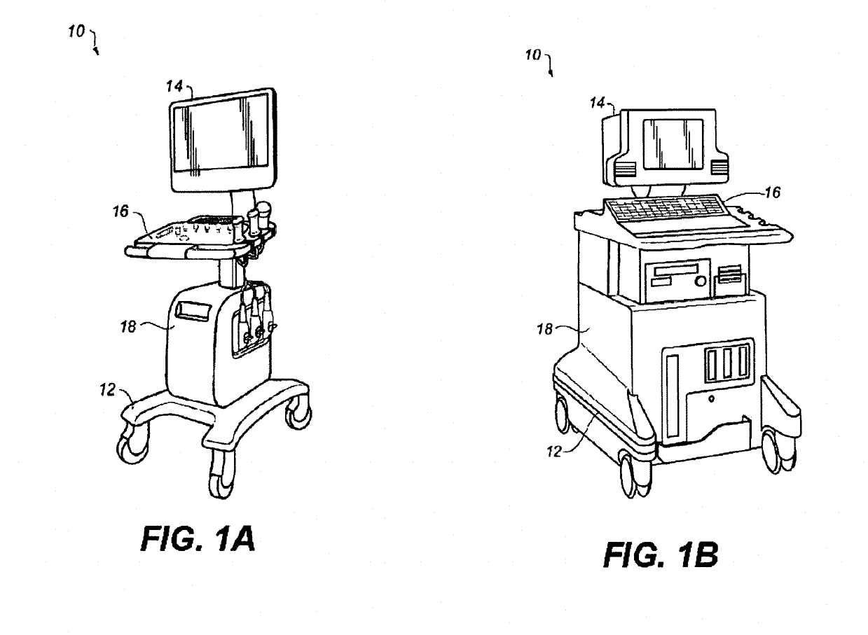

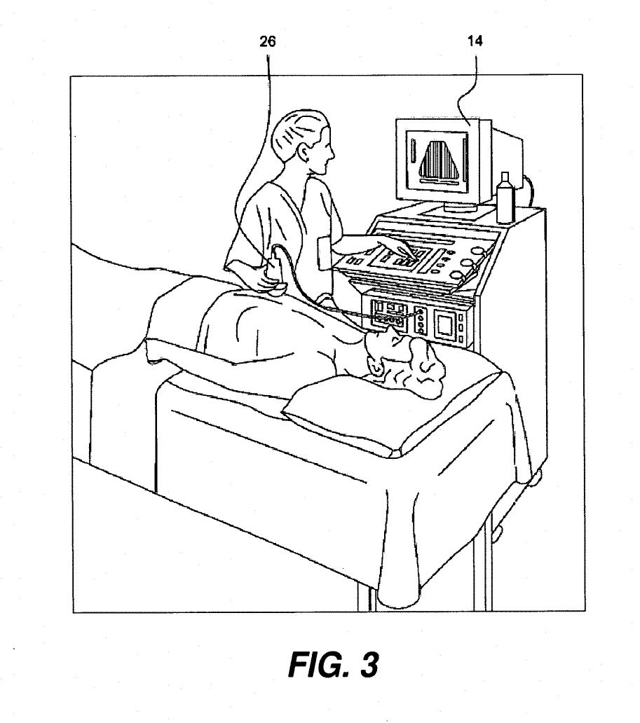

FIGS. 1A, 1B, 2, and 3 show exemplary ultrasound systems 10 including a cart/base/support 12, a display monitor 14, an input device (such as keyboard 16 or mouse), and a generator 18. The display 14 can be a touchscreen to function as an input device. As illustrated, the ultrasound system is a mobile system having wheels. FIG. 3 shows an example of an ultrasound image displayed on the display 14.

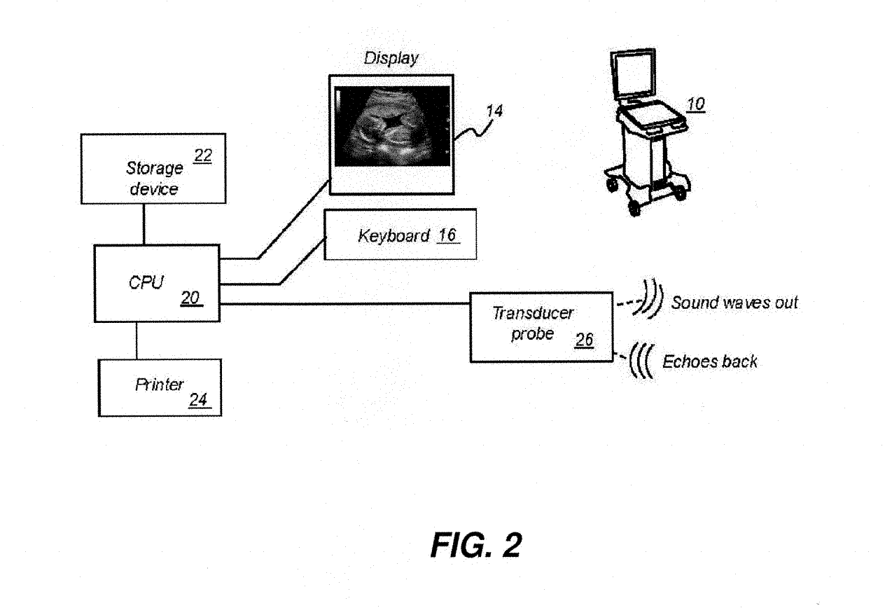

As FIG. 2 shows, the ultrasound system 10 has a central processing unit CPU 20, a control logic processor that provides control signals and processing capabilities. CPU 20 is in signal communication with display 14 and interface device 16, as well as with a storage device 22 and an optional printer 24. A transducer probe 26 provides the ultrasound acoustic signal and generates an electronic feedback signal indicative of tissue characteristics from the echoed sound.

FIG. 3 shows an example of an ultrasound system 10 in use with an image provided on display/monitor 14.

Ultrasound is sound wave energy with frequencies higher than those audible to the human ear. Ultrasonic images, also known as sonograms, are made by directing pulses of ultrasound into tissue using a probe. The sound echoes off the tissue; with different tissues reflecting varying degrees of sound. These echoes are recorded and displayed as an image to the operator.

Different types of images can be formed using sonographic instruments. The most well-known type is a B-mode image, which displays the acoustic impedance of a two-dimensional cross-section of tissue. Other types of image can display blood flow, motion of tissue over time, the location of blood, the presence of specific materials, the stiffness of tissue, or the anatomy of a three-dimensional region.

Accordingly, the system of FIGS. 1A and 1B is configured to operate within at least two different ultrasound modes. As such, the system provides means to switch between the at least two different ultrasound modes. Such a two-mode configuration and means for switching between modes are well known within the ultrasound technology.

Clinical modes of ultrasound used in medical imaging generally include the following:

A-mode: A-mode (amplitude mode) is the simplest type of ultrasound. A single transducer scans a line through the body with the echoes plotted on screen as a function of depth. Therapeutic ultrasound aimed at a specific tumor or calculus also uses A-mode emission to allow for pinpoint accurate focus of the destructive wave energy.

B-mode or 2D mode: In B-mode (brightness mode) ultrasound, a linear array of transducers simultaneously scans a plane through the body that can be viewed as a two-dimensional image on screen. Sometimes referred to as 2D mode, this mode is effective for showing positional and dimensional characteristics of internal structures and is generally the starting point for exam types that use other modes.

C-mode: A C-mode image is formed in a plane normal to a B-mode image. A gate that selects data from a specific depth from an A-mode line is used; the transducer is moved in the 2D plane to sample the entire region at this fixed depth. When the transducer traverses the area in a spiral, an area of 100 cm² can be scanned in around 10 seconds.

M-mode: In M-mode (motion mode) ultrasound, pulses are emitted in quick succession. With each pulse, either an A-mode or B-mode image is acquired. Over time, M-mode imaging is analogous to recording a video in ultrasound. As the organ boundaries that produce reflections move relative to the probe, this mode can be used to determine the velocity of specific organ structures.

Doppler mode: This mode makes use of the Doppler effect in measuring and visualizing blood flow.

Color Doppler: Velocity information is presented as a color-coded overlay on top of a B-mode image. This mode is sometimes referred to as Color Flow or color mode.

Continuous Doppler: Doppler information is sampled along a line through the body, and all velocities detected at each point in time are presented (on a time line).

Pulsed wave (PW) Doppler: Doppler information is sampled from only a small sample volume (defined in 2D image), and presented on a timeline.

Duplex: a common name for the simultaneous presentation of 2D and (usually) PW Doppler information. (Using modern ultrasound machines, color Doppler is almost always also used; hence the alternative name Triplex.).

Pulse inversion mode: In this mode, two successive pulses with opposite sign are emitted and then subtracted from each other. This implies that any linearly responding constituent will disappear while gases with non-linear compressibility stand out. Pulse inversion may also be used in a similar manner as in Harmonic mode.

Harmonic mode: In this mode a deep penetrating fundamental frequency is emitted into the body and a harmonic overtone is detected. With this method, noise and artifacts due to reverberation and aberration are greatly reduced. Some also believe that penetration depth can be gained with improved lateral resolution; however, this is not well documented.

A Sonographer, ultrasonographer, clinician, practitioner, or other clinical user, is a healthcare professional (often a radiographer but may be any healthcare professional with the appropriate training) who specializes in the use of ultrasonic imaging devices to produce diagnostic images, scans, videos, or 3D volumes of anatomy and diagnostic data.

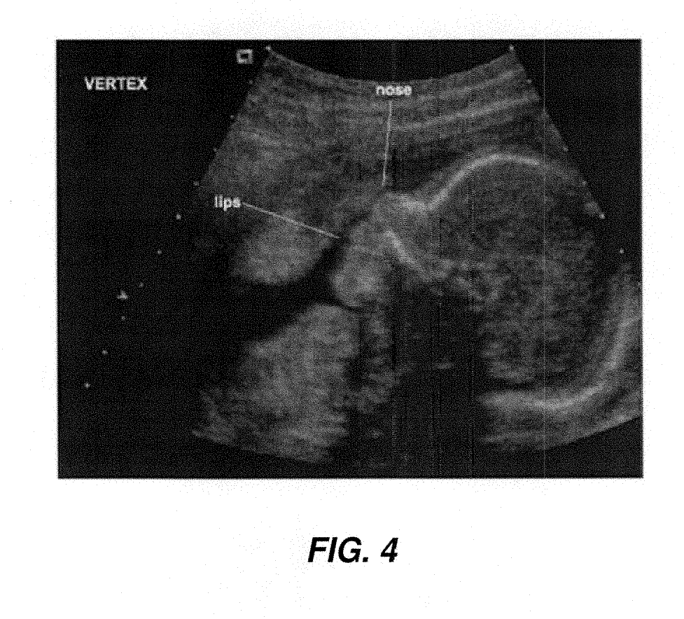

FIG. 4 shows a displayed ultrasound image in grey scale. Such an image would be captured using a grey scale mode, for example, using B-mode.

As noted previously, various beamforming algorithms are available on ultrasound systems. Embodiments of the present disclosure employ a closed-loop data-driven approach to selecting the beamforming algorithm or paradigm based on a priori knowledge of the physics of a mode, or based on the data obtained during a certain acquisition, or based on a combination of both.

It is understood that the different beamforming types and associated imaging algorithms have advantages as well as limitations. For example, diverging beams and plane wave beamforming signals provide high frame rates, but do not, in conventional implementations, provide focus for the transmitted signal, which can result in poorer spatial and contrast resolution. High frame rates are achievable with these beamforming signal sequences because they allow reconstruction over larger regions with relatively fewer number of signal emissions. In the context of the present disclosure, the phrase “diverging beam/plane wave” is used to represent emission using either divergent or plane beamforming types, since there is often no formal distinction needed between the types.

Recently, a number of novel transmission and signal processing schemes have been developed and reported in the literature, describing methods to overcome fundamental shortcomings of plane wave and divergent beam imaging. For example, coherent plane wave compounding has been introduced, wherein the plane waves are transmitted into tissue from multiple overlapping directions and are coherently summed to improve the image resolution by reducing side lobes. Conversely, delay-sum beamforming or more conventional beamforming schemes allow tightly focused beams, and therefore provide better spatial resolution for a targeted region of interest (ROI), but at lower frame rates. More particularly, some modes such as harmonic imaging, while feasible, are more challenging with diverging and plane wave beams.

Harmonic imaging relies on imaging the harmonic signal components that are generated as the incident acoustic wave propagates through the tissue due to the non-linear properties of tissue. The higher the acoustic pressure in the tissue, the more pronounced the harmonic signal content. Since conventional beamforming types typically rely on narrowly focused transmit signals, they are inherently better suited for harmonic imaging, when compared to plane wave and diverging beam approaches. However, there can be a number of instances in which plane wave and diverging beam beamforming signal types can be advantaged. To help improve system operability, embodiments of the present disclosure address the problem of beamforming selection by beginning a scan using a first beamforming type, analyzing the data generated following the first beamforming paradigm as this data is generated, and dynamically determining whether or not to switch to another beamforming type. A novel aspect of the present disclosure is a proposed method wherein the optimal beamforming type for the specific clinical environment (considering factors such as anatomy, body habitus, and ultrasound system capability) is automatically selected, or is suggested to the operator, in order to provide a useful clinical outcome.

Diverging beam and plane wave beamforming allow high temporal resolution color flow data to be acquired. The same data, saved over a short time period, can be used to create a Spectral Doppler waveform. Advantageously, the acquired data is available over the entire image rather than at only a single location.

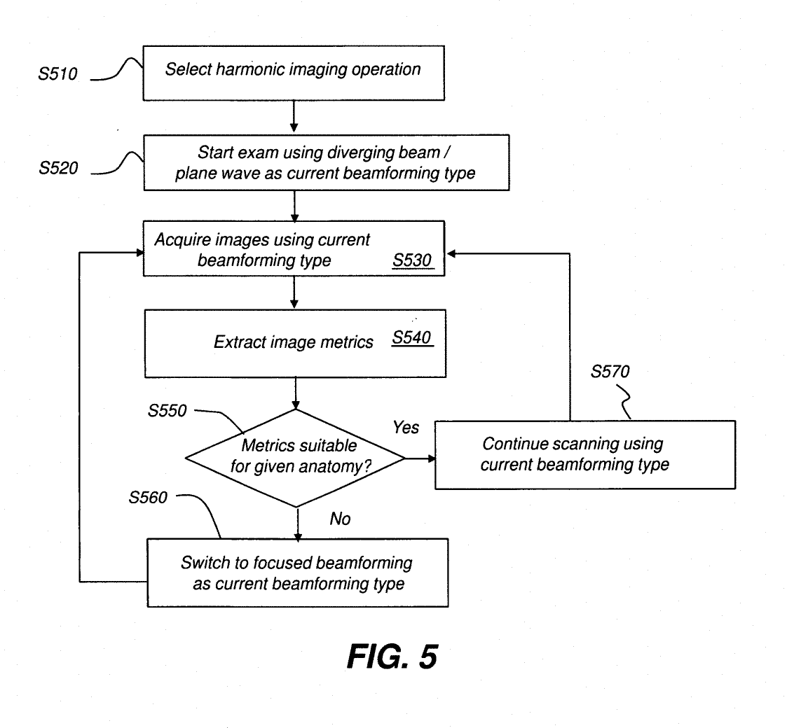

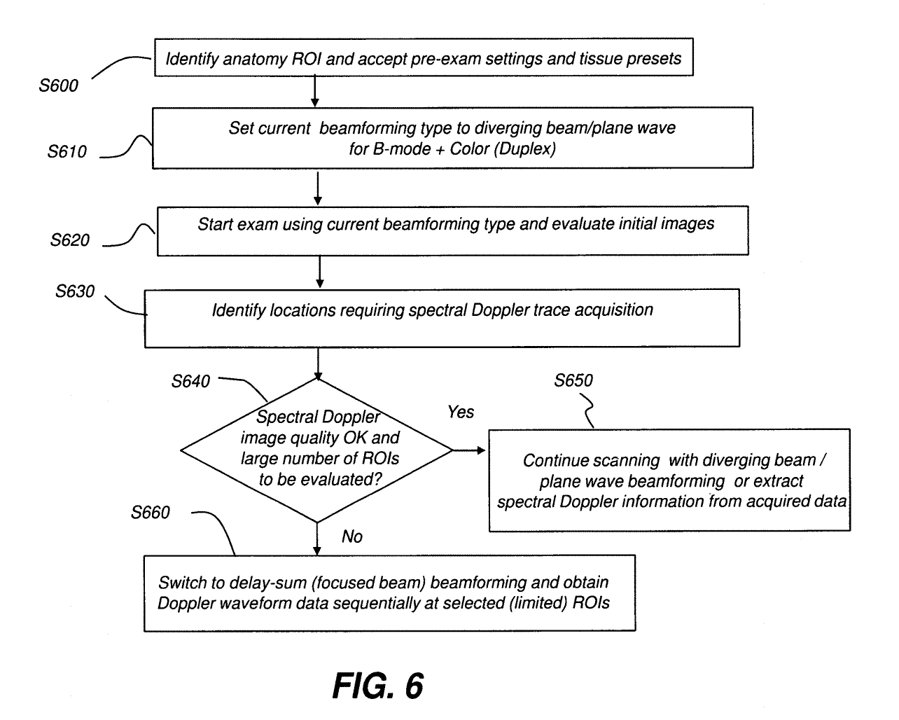

Referring to FIGS. 5 and 6, there are shown flow charts for system operation according to an embodiment of the present disclosure. In classic duplex imaging, the goal or intent is to obtain B-mode, Color Flow, and/or Spectral Doppler information. Another scenario relates to performing harmonic imaging. In both these imaging modes, it would be desirable to have the ability to switch the beamforming paradigm during the exam for improved focus on different aspects of information to be obtained from the ultrasound exam.

FIG. 5 shows a flowchart of operational steps for tissue harmonic imaging. As shown, a clinical ultrasound exam is initiated, with the ultrasound system set to operate using a diverging beam/plane wave beamforming paradigm. An initial set of images is acquired in a harmonic imaging mode. A data analysis is performed to extract at least one image metric (preferably, a key image metric) with information such as contrast resolution, spatial resolution, penetration, frame rate, and the like. A determination is made as to whether or not the extracted metrics are suitable, such as within an acceptable range of values, for the given anatomy and body part. If the metrics are acceptable, scanning can continue using the same diverging beam/plane wave beamforming signal type. If the metrics are not suitable, it can be beneficial to switch to the delay-sum (focused beam) beamforming signal type or to another beamforming variant suitable for harmonic imaging. Suitability can be determined by comparing the values of the metrics against one or more stored threshold values, for example.

In the sequence shown in FIG. 5, an initial setting step S510 sets the current system mode controls for harmonic imaging. A scan initiation step S520 then starts scanning for the exam using diverging beam or plane wave beamforming as the current beamforming type for acquiring an initial set of images to begin the scan process. An acquisition step S530 continues to acquire ultrasound images using the current beamforming type. An extraction step S540 then performs the data analysis that extracts useful image metrics, such as contrast, resolution, spatial resolution, penetration data, and frame rate information from the acquisition process of step S530. To evaluate harmonic mode performance, the spectral content (i.e. signal power) in the harmonic frequency band could also be compared with the signal power in the fundamental frequency band.

Continuing with the sequence of FIG. 5, a decision step S550 determines whether or not the extracted metrics calculated/determined/extracted from step S540 are suitable for the given anatomy and body part of the region of interest. These metrics can be based on statistical data and thresholds from phantom and other imaging knowledge, according to known a priori information about the scanned anatomy and other exam-specific factors, such as patient age, size, sex, and other data. If metrics are satisfactory, a continuation step S570 executes, with no change to beamforming type, returning to image acquisition and optionally repeating steps S530, S540, and S550 as many times as desired. Alternatively, if subsequent metrics show need for a change in current beamforming type during operation, a beamforming type change step S560 executes, suggesting a change, such as providing a message on the display monitor, or automatically changing the beamforming type to one that is more appropriate for the given anatomy and region of interest (ROI). This change may be to delay-sum (focused beam) beamforming or to some other variant suitable for harmonic imaging, for example. It should be noted that, as an alternative to automatic beamforming type switching, operator override may be provided, maintaining the current beamforming type or specifying another beamforming type to serve as the current beamforming type.

Optionally, instead of switching the beamforming type, the system could automatically adjust filter settings for the current beamforming type, based on the harmonic frequency analysis. It should be noted that automated execution of beamforming type change step S560 can be optional; as an alternative, a message or prompt is provided to the operator allowing entry, confirmation, or override of the proposed change to the current beamforming type.

FIG. 6 shows a flowchart of duplex imaging and spectral Doppler evaluation. In an initial exam data acquisition step S600, information related to the specific exam is acquired. This can include such information as the anatomy and ROI for the exam, and any pre-exam settings and tissue settings, such as from the exam protocol, for example. As shown, a clinical ultrasound exam can be initiated with diverging beam/plane wave beamforming paradigm in B-mode+Color (Duplex), allowing acquisition of image content that provides a scout image. An initial set of images is acquired and the anatomy is evaluated.

For the FIG. 6 sequence, in a beamforming type specification step S610, the system is set to diverging or plane wave beamforming type. Since plane wave beamforming provides favorable frame rates, it is an advantaged beamforming type for generating one or more scout images. Moreover, as has been more recently demonstrated, the high frame rate Color Flow data that has been acquired with plane wave or diverging beams can also be used to retrospectively obtain Spectral Doppler information, extracted from a stored sequence of images. Based on such a retrospective analysis, locations for acquiring spectral Doppler image content can be readily identified. In addition, a determination is also made as to whether the Spectral Doppler image quality is satisfactory and whether there are a large number of ROIs to be evaluated, exceeding a predetermined threshold number. If yes, execution continues with the diverging beam/plane wave beamforming approach. If not, then the system can switch to delay-sum (focused beam) beamforming and obtain Doppler waveform data sequentially at a select (limited) number of ROIs.

The sequence shown in FIG. 6 shows a series of steps for duplex imaging and spectral Doppler evaluation according to an embodiment, with optional acquisition and use of a scout image. The scout image, as used in various radiographic imaging applications, can provide a coarse-resolution view of the scanned region as an aid to the sonographer, providing visual guidance for positioning the transducer probe. In beamforming type specification step S610, the system is set to generate a diverging or plane wave beamforming type. This sets up the system for generating a diverging beam or plane wave signal to provide B-mode imaging. Advantageously, the diverging beam or plane wave signal is advantaged for acquisition speed, processing, and analysis. The diverging or plane wave beamforming type is then, temporarily, the current beamforming type.

A scan initiation step S620 starts the exam using the specified beamforming type, acquiring images and evaluating the imaged anatomy. An identification step S630 then retrospectively reviews the acquired high frame rate data to visualize flow information equivalent to what is obtained with spectral Doppler and determines how many ROIs must be evaluated using spectral Doppler imaging. This provides information appropriate for the specific exam, anatomy, and patient condition and can be influenced by system guidance factors such as established protocols or guidance from machine learning algorithms provided with the control software for the imaging apparatus. Useful information can include, for example, ROIs including veins or arteries. Spectral Doppler imaging can be performed using plane wave beamforming, without the need for the operator to switch to that beamforming type.

A decision step S640 determines whether or not the spectral Doppler image quality using plane wave beamforming is satisfactory and whether or not there are a large number of ROIs to be evaluated, exceeding a predetermined threshold value. If so, a continuation step S650 continues scanning using diverging beam/plane wave beamforming. If there are only a small number of ROIs for Doppler imaging or the retrospectively reviewed Spectral Doppler image quality (from the plane wave/diverging beam scheme) is disappointing, a switching step S660 executes, switching the system to conventional delay-sum (focused beam) beamforming. Switching step S660 can be suggested to the operator for confirmation or other explicit response, or can be automatically executed, subject to operator override, for example. The operator can terminate the image acquisition and evaluation at any suitable time.

In a typical workflow, the system can be initially set up for Duplex operation, scanning using planewave/diverging beam beamforming (software beamforming) type for high frame rate acquisition. In this process, the complete evaluation of the organ (such as via an optional scout scan) is performed. B-mode and Color Flow information is obtained in this process. The optional scout scan can be used to help locate the region of interest (ROI). Since the frame rates for planewave/diverging beam beamforming types are higher (in some situations, significantly higher than with conventional focused beam types), a large amount of information (including data required for Spectral Doppler generation) can be available in a short time. This data from the ultrasound system can be quickly evaluated by the practitioner or sonographer to determine the appropriate region of interest. For instance, the locations proximal, distal and at the stenosis can be readily determined in a carotid scan.

Once the initial or scout scan is performed, the beamforming type can be switched in the system software. In an embodiment, these changes can be performed without user knowledge or interaction. Optionally, clinical protocol or operator preference may dictate that spectral Doppler imaging using focused beamforming be used for quantifying the flow information. Also, based on the physics of signal delivery and acquisition, the focused beam approach with conventional delay-sum beamforming has the ability to provide high spatial resolution Doppler traces. As such, in some situations it is advantageous to change the beamforming type to more conventional delay-sum beamforming for obtaining high fidelity data in the region of interest for clinical decision making.

Another embodiment involves the use of tissue harmonic imaging with the most prudent beamforming choice. For example, the ultrasound exam could begin using a first beamforming type (typically diverging beam/plane wave beamforming). During the initial phase of the scan, the data is evaluated to determine important characteristics such as penetration, contrast resolution, spatial resolution, and harmonic content. Since these are affected by tissue specific characteristics, performing on-the-fly analysis and making decisions on the beamforming approach to use in the remaining portion of the exam is very valuable. For example, after the initial scan, if the acoustic signal penetration is deemed inadequate, the system could automatically switch to a delay sum beamforming type, resulting in a tradeoff that employs reduced frame rate but provides improved resolution which might be more valuable for the user. On the other hand, if the resolution is adequate based on the data analysis, the system could continue to operate in the same beamforming paradigm providing the continued benefit of improved frame rate to the user.

Accordingly, Applicants have described an first ultrasound imaging method, comprising: (1) initiating an ultrasound exam on an anatomy; (2) acquiring a first set of images using harmonic imaging; (3) performing a data analysis to extract at least one image metric; (4) determining whether the at least one image metric is suitable for the anatomy and/or body part; (5) if the at least one image metric is determined to be suitable, scanning by employing a plane wave or divergent beam beamforming type; and (6) if the at least one image metric is determined to be unsuitable, switching to a delay-sum (focused beam) beamforming type or other variant suited for harmonic imaging.

In a particular arrangement of this method, the initiating an ultrasound exam on an anatomy is accomplished by employing a diverging beam/plane wave beamforming type.

In a particular arrangement of this method, the image metrics includes one of the following: contrast resolution, spatial resolution, penetration, and frame rate, and harmonic energy content (absolute or relative to the fundamental energy).

In a particular arrangement of this method, the at least one image metric is determined to be suitable or unsuitable by comparing the at least one metric to a pre-determined threshold metric.

Accordingly, Applicants have described an first ultrasound imaging method, comprising: (a) initiating an ultrasound exam on an anatomy by employing diverging beam/plane wave beamforming type in B-mode+Color (Duplex) operation; (b) acquiring a first set of images; (c) evaluating the anatomy of at least one of the first set of images; (d) in response to the anatomy evaluation, identifying at least one location for a spectral Doppler trace; (e) assessing image quality of at least one spectral Doppler trace; (f) if the image quality is determined to be suitable, scanning by employing the diverging beam/plane wave beamforming type; and (g) if the image quality is determined to be unsuitable, scanning by employing a delay-sum (focused beam) beamforming type or other variant suited for harmonic imaging.

Applicants have disclosed a method for ultrasound imaging that acquires an initial set of one or more images of a region of interest in a predetermined clinical ultrasound mode using a generated signal of a first beamforming type. The method further consists in evaluating the suitability of the first beamforming type signal for imaging the region of interest in the predetermined mode according to a metric calculated/determined/extracted from the initial set of one or more images. The method can automatically change the generated signal to a second beamforming type according to the suitability evaluation. Alternately, a change to a second beamforming type can be recommended, subject to operator instruction.

In the detailed description provided herein, the example of harmonic mode was described, since this clinical mode can work well using either focused or plane wave beamforming signal types, depending in part on tissue characteristics. However, it should be noted that the process described herein can be used with other clinical modes, as well, such as for elastography mode imaging for example. Thus, with the ultrasound system setup for imaging in an elastography mode, the initial beamforming signal type can be set for plane wave beamforming. Then, following evaluation of the suitability of plane wave beamforming, the system may automatically change to a focused beam or to some other appropriate beamforming signal.

In a particular arrangement of this method, the image quality is determined to be suitable or unsuitable by providing a pre-determined threshold image quality metric.

The present invention can be a software program. Those skilled in the art will recognize that the equivalent of such software may also be constructed in hardware. Because image manipulation algorithms and systems are well known, the present description will be directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A method for ultrasound imaging, comprising:
acquiring an initial set of one or more images of a region of interest in a predetermined clinical ultrasound mode using a first generated signal of a first beamforming type;
evaluating the suitability of the first generated signal for imaging the region of interest in the predetermined mode according to a image metric determined from the initial set of one or more images; and
automatically changing the first generated signal to a second generated signal of a second beamforming type according to the suitability evaluation.

2. The method of claim 1 wherein the first beamforming type employs a diverging beam/plane wave signal.

3. The method of claim 1 wherein the image metric includes one or more of the following: contrast resolution, spatial resolution, penetration, power in harmonic frequency band or ratio of power between harmonic and fundamental frequency bands, and frame rate.

4. The method of claim 1 wherein the image metric is determined to be suitable or unsuitable by comparison with a stored threshold metric.

5. The method of claim 1 wherein the step of evaluating is performed automatically, without operator intervention.

6. The method of claim 1 wherein the step of automatically changing the generated signal can be overridden by a user instruction.

7. The method of claim 1 wherein the predetermined clinical ultrasound mode is a harmonic imaging mode.

8. An ultrasound method, comprising:
initiating an ultrasound exam on an anatomy;
acquiring a first set of images of the anatomy using a harmonic imaging mode;
performing a data analysis to extract at least one image metric;
determining whether the at least one image metric is suitable for the anatomy;
if the at least one image metric is suitable, scanning the anatomy by employing plane wave or divergent beam beamforming as a current beamforming type; and
if the at least one image metric is unsuitable, switching to a delay-sum or focused beam beamforming type or other beamforming signal variant for harmonic imaging.

9. The method of claim 8 wherein the step of initiating the ultrasound exam is accomplished by employing a diverging beam/plane wave beamforming paradigm.

10. The method of claim 8 wherein the image metrics includes one or more of the following: contrast resolution, spatial resolution, penetration, power in harmonic frequency band or ratio of power between harmonic and fundamental frequency bands, and frame rate.

11. The method of claim 8 wherein the step of determining whether the at least one image metric is suitable or unsuitable is accomplished by comparing the at least one metric to a pre-determined threshold metric.

12. The method of claim 8 wherein the step of determining whether the at least one image metric is suitable or unsuitable is accomplished automatically, without operator intervention.

13. An ultrasound method, comprising:
initiating an ultrasound exam of anatomy for B-mode+Color (Duplex) operation, employing a diverging beam or plane wave beamforming signal type and acquiring a first set of images;
evaluating the anatomy of at least one of the first set of images;
in response to the anatomy evaluation, identifying at least one location for a spectral Doppler trace;
assessing an image quality of at least one spectral Doppler trace;
if the image quality is assessed as suitable, scanning by employing the same diverging beam or plane wave beamforming signal type; and
if the image quality is assessed as unsuitable, scanning by changing to a delay-sum (focused beam) beamforming type or other beamforming signal variant suited for harmonic imaging.

14. The method of claim 13 wherein the image quality is assessed to be suitable or unsuitable according to a pre-determined threshold image quality.

15. The method of claim 13 further comprising automatically adjusting one or more filter values according to image quality suitability.
